(19) United States
(12) Patent Application Publication
(10) Pub. No.: US 2023/0062311 A1
(43) Pub. Date: Mar. 2, 2023

(54) SPORTS INJURY SENSING SYSTEM AND METHOD

(71) Applicant: INSTITUTE FOR INFORMATION INDUSTRY, TAIPEI (TW)

(72) Inventors: Zhi Ying CHEN, Taipei (TW); Hsun-Hui HUANG, Taipei (TW); Chia Hao WANG, Taipei (TW); Shr Yau JAN, Taipei (TW); Chien-Der LIN, Taipei (TW)

(21) Appl. No.: 17/450,997

(22) Filed: Oct. 15, 2021

(30) Foreign Application Priority Data
Aug. 31, 2021 (TW) 110132340

(51) Int. Cl. A61B 5/0205; A61B 5/01; A61B 5/11; A61B 5/00; A61B 5/024

(52) U.S. Cl. CPC A61B 5/02055; A61B 5/015; A61B 5/1126; A61B 5/1122; A61B 5/746; A61B 5/1118; A61B 5/024; A61B 2562/0219; A61B 2562/04

(57) ABSTRACT

A sports injury sensing system and method are provided. The sports injury sensing system includes a sports injury sensing apparatus. The apparatus receives a plurality of inertial sensing data of a user, and each of the inertial sensing data corresponds to a body part of the user. The apparatus determines an exercising body part of the user based on the inertial sensing data. The apparatus receives a thermal image sensing datum corresponding to the exercising body part, wherein the thermal image sensing datum indicates a body temperature state of the user. The apparatus analyzes a temperature change of surrounding muscles of the exercising body part based on the thermal image sensing datum. The apparatus calculates a sports injury assessment based on the inertial sensing data and the temperature change of surrounding muscles.

### CROSS-REFERENCE TO RELATED APPLICATION

This application claims priority to Taiwan Application Serial Number 110132340, filed Aug. 31, 2021, which is herein incorporated by reference in its entirety.

### BACKGROUND

### Field of Invention

The present invention relates to a sports injury sensing system and method. More particularly, the present invention relates to a system and method for evaluating sports injuries by analyzing inertial sensing data and thermal image sensing datum.

### Description of Related Art

In recent years, the trend of sports has become more and more popular, and people have paid more attention to various sports needs. To improve exercise efficiency, there is an urgent need to assist in reviewing the physiology and physical fitness of athletes through the collection and analysis of physical fitness data, the purpose is to ensure that the amount of training is appropriate and will not cause an injury.

In traditional training methods, “sports injuries” caused by wrong exercise postures or overloaded training are usually not easy to be detected immediately. Even an experienced coach can hardly determine in real-time whether the athlete's muscle strength and training content are within a reasonable training range during the training. Therefore, it is usually necessary to wait a few days (e.g., three to five days) to observe the recovery of muscle soreness before knowing whether it is a sports injury.

Accordingly, there is an urgent need for a technique that can provide a sports injury sensing technology that can instantly generate a warning or a sports injury assessment based on the user's exercise status.

### SUMMARY

An objective of the present invention is to provide a sports injury sensing system. The sports injury sensing system comprises a sports injury sensing apparatus. The sports injury sensing apparatus receives a plurality of inertial sensing data of a user, and each of the inertial sensing data corresponds to a body part of the user. The sports injury sensing apparatus determines an exercising body part of the user based on the inertial sensing data. The sports injury sensing apparatus receives a thermal image sensing datum corresponding to the exercising body part, and the thermal image sensing datum indicates a body temperature state of the user. The sports injury sensing apparatus analyzes a temperature change of surrounding muscles of the exercising body part based on the thermal image sensing datum. The sports injury sensing apparatus calculates a sports injury assessment based on the inertial sensing data and the temperature change of surrounding muscles.

Another objective of the present invention is to provide a sports injury sensing method, which is adapted for use in a sports injury sensing system. The sports injury sensing system comprises a sports injury sensing apparatus. The sports injury sensing method is performed by the sports injury sensing apparatus and comprises following steps: (a) receiving a plurality of inertial sensing data of a user, wherein each of the inertial sensing data corresponds to a body part of the user, (b) determining an exercising body part of the user based on the inertial sensing data, (c) receiving a thermal image sensing datum corresponding to the exercising body part, wherein the thermal image sensing datum indicates a body temperature state of the user, (d) analyzing a temperature change of surrounding muscles of the exercising body part based on the thermal image sensing datum, and (e) calculating a sports injury assessment based on the inertial sensing data and the temperature change of surrounding muscles.

According to the above descriptions, the sports injury sensing technology (including the system and the method) provided by the present invention collects a plurality of inertial sensing data through a plurality of inertial sensors installed on a body of a user, determines an exercising body part (e.g., a body part that is being frequently exercised) of the user based on the inertial sensing data, analyzes a temperature change of surrounding muscles of the exercising body part based on the thermal image sensing datum, calculates a sports injury assessment based on the inertial sensing data and the temperature change of surrounding muscles. The sports injury sensing technology provided by the present invention analyzes the inertial sensing data and the thermal image sensing datum to determine possible sports injuries in advance, and generates a danger warning message and sports injury assessments to assist users in training and solve the problem in the prior art that the conventional technology cannot immediately assist users in discovering possible sports injuries.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a schematic view depicting an applicable scenarios of a sports injury sensing system of the first embodiment;

FIG. 2 is a schematic view depicting a sports injury sensing apparatus of the first embodiment; and;

FIG. 3 is a partial flowchart depicting a sports injury sensing method of the second embodiment.

### DETAILED DESCRIPTION

In the following description, a sports injury sensing system and method according to the present invention will be explained with reference to embodiments thereof. However, these embodiments are not intended to limit the present invention to any environment, applications, or implementations described in these embodiments. Therefore, description of these embodiments is only for purpose of illustration rather than to limit the present invention. It shall be appreciated that, in the following embodiments and the attached drawings, elements unrelated to the present invention are omitted from depiction. In addition, dimensions of individual elements and dimensional relationships among individual elements in the attached drawings are provided only for illustration but not to limit the scope of the present invention.

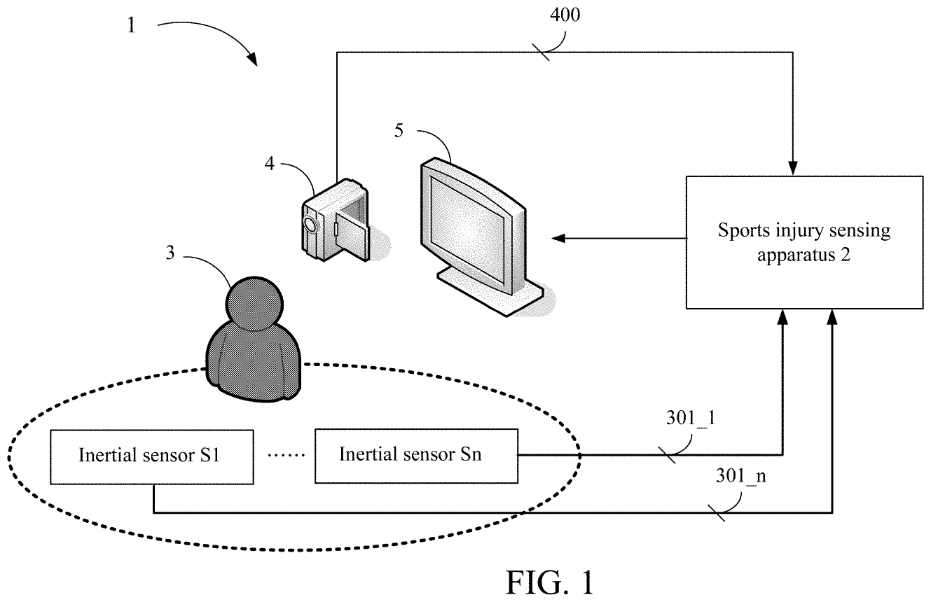

First, an applicable scenario of the present embodiment will be explained, and a schematic view is depicted in FIG. 1. As shown in FIG. 1, in the first embodiment of the present invention, the sports injury sensing system 1 comprises a sports injury sensing apparatus 2. In this scenario, the user 3 wears a plurality of inertial sensors S1, . . . , Sn to exercise, where n is a positive integer greater than 2. The sports injury sensing apparatus 2 can be connected to the inertial sensors S1, . . . , Sn through a wired network or a wireless network. The inertial sensors S1, . . . , Sn are used to continuously generate inertial sensing data 301_1, . . . , 301_n (e.g., at a frequency of 10 times per second), and transmit the inertial sensing data 301_1, . . . , 301_n to the sports injury sensing apparatus 2.

It shall be appreciated that the inertial sensing data 301_1, . . . , 301_n generated by the inertial sensors S1, . . . , Sn at least comprise three-axis speed, angular velocity, and magnetic force. The inertial sensors S1, . . . , Sn can be wearable inertial sensors, which are convenient to be fixed on the body part of the user 3. For example, if the present exercise is mainly for training the lower limbs, the user 3 can set the inertial sensors S1, . . . , Sn on the body part of thighs, calves, knee joints, ankle joints, and hip joints for data collection.

As shown in FIG. 1, an infrared thermal imager sensor 4 can be installed near the user 3 to facilitate sensing a body temperature state of the user 3. The sports injury sensing apparatus 2 can be connected to the infrared thermal imager sensor 4 through a wired network or a wireless network. The infrared thermal imager sensor 4 is used to generate a thermal image sensing datum 400 and transmit the thermal image sensing datum 400 to the sports injury sensing apparatus 2, and the thermal image sensing datum 400 can display the body temperature in different body parts of the user 3.

In some embodiments, the infrared thermal imager sensor 4 can only sense a certain body part of the user 3, such as the lower body part. It shall be appreciated that the infrared thermal imager sensor 4 can transmit the data through interfaces such as MIPI, SPI, I2C, etc. The corresponding operations shall be appreciated by those of ordinary skill in the art and thus will not be further described herein.

As shown in FIG. 1, the sports injury sensing apparatus 2 can be connected to other external apparatus via a wired network or a wireless network, such as a display screen 5 (e.g., the display screen 5 can be used to display information/notify to the user 3). In some embodiments, the sports injury sensing apparatus 2 and the display screen 5 may be provided on the same apparatus, such as a smart phone. It shall be appreciated that, FIG. 1 is only used as an example, and the present invention does not limit the content of the sports injury sensing system 1. For example, the present invention does not limit the number of external apparatus connected to the sports injury sensing apparatus 2. The sports injury sensing apparatus 2 can simultaneously connect to multiple inertial sensors and multiple infrared thermal imager sensors through the network, and it depends on the scale and actual requirements of the sports injury sensing system 1.

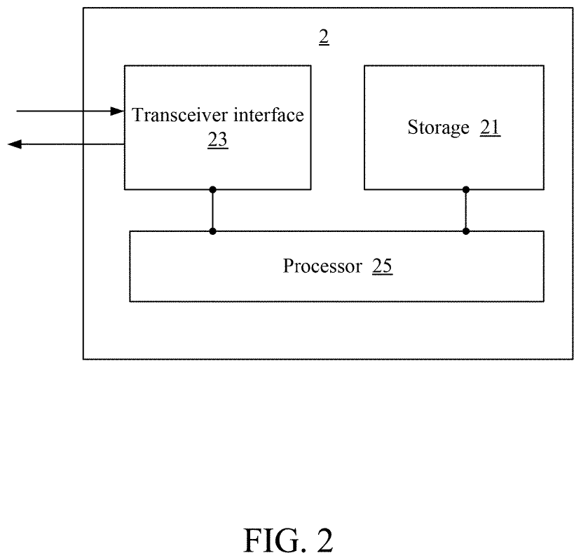

A first embodiment of the present invention is a sports injury sensing apparatus 2 and a schematic view of which is depicted in FIG. 2. The sports injury sensing apparatus 2 comprises a storage 21, a transceiver interface 23 and a processor 25, wherein the processor 25 is electrically connected to the storage 21 and the transceiver interface 23. The storage 21 may be a memory, a Universal Serial Bus (USB) disk, a hard disk, a Compact Disk (CD), a mobile disk, or any other storage medium or circuit known to those of ordinary skill in the art and having the same functionality. The transceiver interface 23 is an interface capable of receiving and transmitting data or other interfaces capable of receiving and transmitting data and known to those of ordinary skill in the art. The transceiver interface 23 can receive data from sources such as external apparatuses, external web pages, external applications, and so on. The processor 25 may be any of various processors, Central Processing Units (CPUs), microprocessors, digital signal processors or other computing apparatuses known to those of ordinary skill in the art.

In some embodiments, the sports injury sensing apparatus 2 can be, but not limited to, an electronic apparatus such as a wearable apparatus, a mobile electronic apparatus, a desktop computer, a portable computer. For example, the sports injury sensing apparatus 2 can be a smart phone, a tablet computer, a smart watch, or other apparatus with networking functions and basic computing capabilities.

The following paragraphs will describe the detailed operations of the first embodiment, please refer to FIG. 1 and FIG. 2. In the present embodiment, the inertial sensors S1, Sn are installed on the body of the user 3 who is in an exercise state, and the inertial sensors S1, Sn generate inertial sensing data 301_1, . . . , 301_n. First, the sports injury sensing apparatus 2 receives the inertial sensing data 201_1, . . . , 201_n from the inertial sensors S1, . . . , Sn, and each of the inertial sensing data 201_1, . . . , 201_n corresponds to one of the body parts of the user 3 (e.g., the body part of thigh, calf, knee joint, ankle joint, hip joint, etc).

Next, the sports injury sensing apparatus 2 determines an exercising body part (e.g., a body part that is being frequently exercised) of the user 3 based on the inertial sensing data 201_1, . . . , 201_n. Specifically, since the inertial sensors S1, Sn can sense the three-axis speed, angular velocity and magnetic force of the position where they are installed (i.e., each body part). Therefore, based on the positional relationship of a plurality of adjacent inertial sensors, the posture of the human body of the user 3 can be estimated, and the angle change of each body part of the user 3 during exercise can be calculated. In the present embodiment, the sports injury sensing apparatus 2 may select a position where the angle changes drastically (i.e., the frequency of the change is fast and the angle change is large) as the exercising body part. For example, if the inertial sensing data generated by the inertial sensors installed on the thighs and lower legs are drastic changes in frequency and amplitude, the sports injury sensing apparatus 2 determines that the knee joints located between the thighs and lower legs are moving frequently.

In some embodiments, the user 3 can pre-select an exercise type and exercise intensity, so that the sports injury sensing apparatus 2 can make a more accurate determination. Specifically, the sports injury sensing apparatus 2 further receives an input datum, wherein the input datum comprises the exercise type and the exercise intensity, and the sports injury sensing apparatus 2 determines the exercising body part based on the input datum and the inertial sensing data. For example, the user 3 can input the present exercise type (e.g., flywheel exercise) and set the exercise intensity to be low, normal, or high intensity. The sports injury sensing apparatus 2 can appropriately adjust a standard of determination based on the exercise type and the exercise intensity.

In some embodiments, in order to prevent the user 3 from experiencing immediate sports injury during exercise, the sports injury sensing apparatus 2 may further use biomechanics (e.g., calculation of force, point of force, angle of force, torque, etc.) and other human body posture calculations to determine in real-time whether it is possible to cause sports injuries based on the inertial sensing data 301_1, . . . , 301_n. If the sports injury sensing apparatus 2 determines that the current posture or movement trajectory of the human body may be dangerous (e.g., bad exercise posture, long-lasting movement, too large joint angle, incorrect position of force during exercise), the sports injury sensing apparatus 2 may immediately issue a notification to warn the user 3. Specifically, the sports injury sensing apparatus 2 can generate a joint angle range and a movement trajectory range based on the exercise type and the exercise intensity. Next, the sports injury sensing apparatus 2 calculates a joint angle and a movement trajectory based on the inertial sensing data. Finally, the sports injury sensing apparatus 2 compares whether the joint angle and the movement trajectory fall within the joint angle range and the movement trajectory range to determine whether to generate a danger warning message.

It shall be appreciated that the aforementioned human body posture calculation can be carried out through the analysis technology of human skeleton state and torque in biomechanics to perform model calculation and analysis of human body posture. Those of ordinary skill in the art shall easily appreciate how to implement based on these descriptions. Therefore, the details will not be repeated herein.

In the present embodiment, the sports injury sensing apparatus 2 further analyzes the temperature change of the exercising body part during exercise (e.g. before and during exercise, or before and after exercise) to detect possible muscle overuse in advance. Specifically, the sports injury sensing apparatus 2 receives the thermal image sensing datum 400 corresponding to the exercising body part, wherein the thermal image sensing datum 400 indicates a body temperature state of the user 3. The sports injury sensing apparatus 2 analyzes a temperature change of surrounding muscles of the exercising body part based on the thermal image sensing datum 400.

For example, when the sports injury sensing apparatus 2 determines that the exercising body part (e.g., a body part that is being frequently exercised) is the knee joint, the sports injury sensing apparatus 2 analyzes the temperature change of the surrounding muscles of the knee joint during exercise. When the temperature change of the surrounding muscles before and during exercise is less than 0.7 degrees, it is a normal state. When the temperature change of the surrounding muscles before and during exercise between 0.8 degrees and 1.5 degrees, it is a state of “to be cautious”. When the temperature change of the surrounding muscles before and during exercise changes more than 1.6 degrees, the exercise is necessary to be stopped immediately.

In some embodiments, in order to prevent the user 3 from experiencing immediate sports injuries during exercise, the sports injury sensing apparatus 2 can evaluate the muscle temperature and determine in real time whether it is possible to cause damage based on the thermal image sensing datum 400. If the sports injury sensing apparatus 2 determines that the current muscle temperature may be dangerous (e.g., excessive muscle temperature difference, muscle overuse, muscle fatigue and inflammation, etc.), the sports injury sensing apparatus 2 may immediately issue a notification to warn the user 3. Specifically, the sports injury sensing apparatus 2 generates a muscle temperature range based on the exercise type and the exercise intensity. Then, the sports injury sensing apparatus 2 compares whether the thermal image sensing datum 400 falls within the muscle temperature range to determine whether to generate a danger warning message.

Finally, after the exercise is over, the sports injury sensing apparatus 2 can further analyze the movement posture and temperature changes of the muscle groups, thereby generating a sports injury assessment, and prevent possible sports injuries in advance by estimating the degree of muscle fatigue (e.g., Delayed onset muscle soreness (DOMS)). Specifically, the exercise injury sensing apparatus 2 calculates a sports injury assessment based on the inertial sensing data 301_1, . . . , 301_n and the temperature change of surrounding muscles.

In some embodiments, the sports injury sensing system 1 may further comprise a heart rate sensor (not shown), the heart rate sensor is connected to the sports injury sensing apparatus 2 through a network. The heart rate sensor is used to sense a heart rate state of the user 3 to generate a heart rate signal and transmit the heart rate signal to the sports injury sensing apparatus 2. In some embodiments, in order to prevent the user 3 from experiencing immediate sports injury during exercise, the sports injury sensing apparatus 2 further determines whether the heart rate state of the user 3 falls within a safe heart rate range according to the heart rate signal, and generates a danger warning message when the heart rate state is not within the safe heart rate range (e.g., the exercise intensity is too high).

In addition, the sports injury sensing apparatus 2 can generate a heart rate curve by measuring the heart rate signal of the user 3, and perform heart rate variability analysis to obtain exercise intensity indicators such as heart rate variability indicators, heart rate recovery rate, physical fitness index, lactate threshold, etc. The sports injury sensing apparatus 2 can generate a danger warning message by the exercise intensity indicators to remind the user 3 to avoid excessive exercise causing the danger of heart and lung.

In some embodiments, the sports injury sensing apparatus 2 may further calculate the sports injury assessment based on the inertial sensing data 301_1, . . . , 301_n, the temperature change of surrounding muscles and the heart rate signal. Specifically, according to the above-mentioned multiple analysis results, the sports injury sensing apparatus 2 analyzes and finds the potential fatigue or wrong posture (e.g., a high temperature difference before and after the thermal image of the muscle group represents a possible muscle strain; or the force should come from the arm, but the measurement shows the wrist is forced, which will cause wrist injury), and outputs the analysis result for warning and the follow-up applications.

In some embodiments, the sports injury sensing apparatus 2 is further connected to a display screen (e.g., the aforementioned display screen 5), and the sports injury sensing apparatus 2 broadcasts posture suggestions and the danger warning message through the display screen to assist the user in training.

According to the above descriptions, the sports injury sensing system provided by the present invention collects a plurality of inertial sensing data through a plurality of inertial sensors installed on a body of a user, determines an exercising body part (e.g., a body part that is being frequently exercised) of the user based on the inertial sensing data, analyzes a temperature change of surrounding muscles of the exercising body part based on the thermal image sensing datum, calculates a sports injury assessment based on the inertial sensing data and the temperature change of surrounding muscles. The sports injury sensing technology provided by the present invention analyzes the inertial sensing data and the thermal image sensing datum to determine possible sports injuries in advance, and generates a danger warning message and sports injury assessments to assist users in training and solve the problem in the prior art that the conventional technology cannot immediately assist users in discovering possible sports injuries.

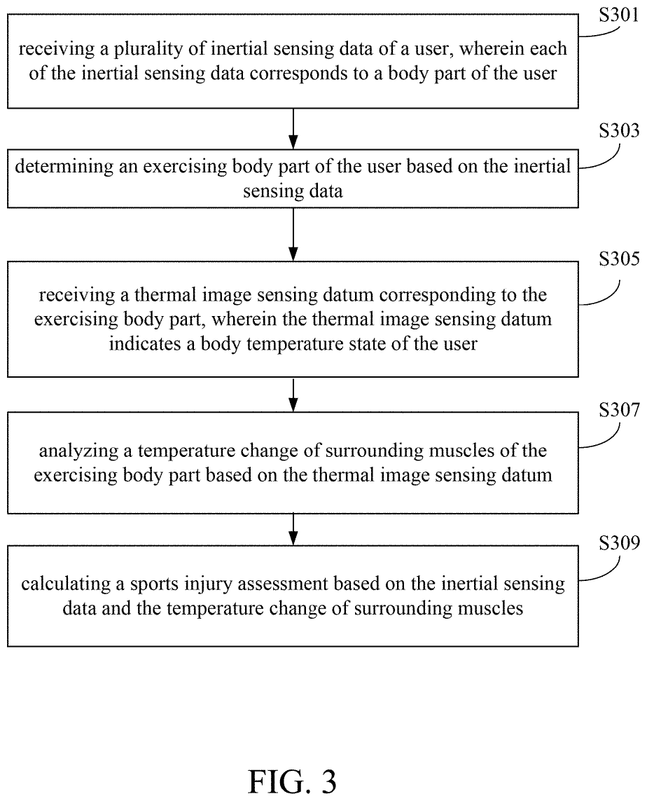

A second embodiment of the present invention is a sports injury sensing method and a flowchart thereof is depicted in FIG. 3. The sports injury sensing method is adapted for a sports injury sensing system, and the sports injury sensing system comprises a sports injury sensing apparatus (e.g., the sports injury sensing system 1 and the sports injury sensing apparatus 2 of the first embodiment). The sports injury sensing method calculates a sports injury assessment through the steps S301 to S309.

In the step S301, the sports injury sensing apparatus receives a plurality of inertial sensing data of a user, wherein each of the inertial sensing data corresponds to a body part of the user. In the step S303, the sports injury sensing apparatus determines an exercising body part of the user based on the inertial sensing data.

In the step S305, the sports injury sensing apparatus receives a thermal image sensing datum corresponding to the exercising body part, wherein the thermal image sensing datum indicates a body temperature state of the user.

In some embodiments, the sports injury sensing system further comprises a plurality of inertial sensors (e.g., the inertial sensors S1, . . . , Sn of the first embodiment) and an infrared thermal imager sensor (e.g., the infrared thermal imager sensor 4 of the first embodiment). The inertial sensors and the infrared thermal imager sensor connect to the sports injury sensing apparatus through a network. Specifically, each inertial sensor is installed on one of the body parts of the user to generate the inertial sensing data of each body part, and the infrared thermal imager sensor is used to generate the thermal image sensing datum.

In the step S307, the sports injury sensing apparatus analyzes a temperature change of surrounding muscles of the exercising body part based on the thermal image sensing datum (e.g., the thermal image sensing datum 400 of the first embodiment). Next, in the step S309, the sports injury sensing apparatus calculates a sports injury assessment based on the inertial sensing data and the temperature change of surrounding muscles.

In some embodiments, the sports injury sensing method further comprises following steps: receiving an input datum, wherein the input datum comprises an exercise type and exercise intensity; and determining the exercising body part based on the input datum and the inertial sensing data.

In some embodiments, the sports injury sensing method further comprises following steps: generating a joint angle range and a movement trajectory range based on the exercise type and the exercise intensity; calculating a joint angle and a movement trajectory based on the inertial sensing data; and comparing whether the joint angle and the movement trajectory fall within the joint angle range and the movement trajectory range to determine whether to generate a danger warning message.

In some embodiments, the sports injury sensing method further comprises following steps: generating a muscle temperature range based on the exercise type and the exercise intensity; and comparing whether the thermal image sensing datum falls within the muscle temperature range to determine whether to generate a danger warning message.

In some embodiments, the sports injury sensing system further comprises a heart rate sensor (e.g., the heart rate sensor of the first embodiment), the heart rate sensor connects to the sports injury sensing apparatus through a network, and the heart rate sensor is used to sense a heart rate state of the user to generate a heart rate signal. Specifically, the sports injury sensing method further comprises following steps: the sports injury sensing apparatus further determining whether the heart rate state of the user falls within a safe heart rate range according to the heart rate signal, and generating a danger warning message when the heart rate state is not within the safe heart rate range.

In addition, the sports injury sensing method further comprises the steps to generate a heart rate curve by measuring the heart rate signal of the user, and perform heart rate variability analysis to obtain exercise intensity indicators such as heart rate variability indicators, heart rate recovery rate, physical fitness index, lactate threshold, etc. The sports injury sensing method further comprises the steps to generate a danger warning message by the exercise intensity indicators to remind the user to avoid excessive exercise causing the danger of heart and lung.

In some embodiments, the sports injury sensing method further comprises following steps: calculating the sports injury assessment based on the inertial sensing data, the temperature change of surrounding muscles and the heart rate signal.

In addition to the aforesaid steps, the second embodiment can also execute all the operations and steps of the sports injury sensing system 1 set forth in the first embodiment, have the same functions, and deliver the same technical effects as the first embodiment. How the second embodiment executes these operations and steps, has the same functions, and delivers the same technical effects will be readily appreciated by those of ordinary skill in the art based on the explanation of the first embodiment. Therefore, the details will not be repeated herein.

According to the above descriptions, the sports injury sensing technology (including the system and the method) provided by the present invention collects a plurality of inertial sensing data through a plurality of inertial sensors installed on a body of a user, determines an exercising body part (e.g., a body part that is being frequently exercised) of the user based on the inertial sensing data, analyzes a temperature change of surrounding muscles of the exercising body part based on the thermal image sensing datum, calculates a sports injury assessment based on the inertial sensing data and the temperature change of surrounding muscles. The sports injury sensing technology provided by the present invention analyzes the inertial sensing data and the thermal image sensing datum to determine possible sports injuries in advance, and generates a danger warning message and sports injury assessments to assist users in training and solve the problem in the prior art that the conventional technology cannot immediately assist users in discovering possible sports injuries.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A sports injury sensing system, comprising:
a sports injury sensing apparatus, being configured to perform following operations:
receiving a plurality of inertial sensing data of a user, wherein each of the inertial sensing data corresponds to a body part of the user;
determining an exercising body part of the user based on the inertial sensing data;
receiving a thermal image sensing datum corresponding to the exercising body part, wherein the thermal image sensing datum indicates a body temperature state of the user;
analyzing a temperature change of surrounding muscles of the exercising body part based on the thermal image sensing datum; and
calculating a sports injury assessment based on the inertial sensing data and the temperature change of surrounding muscles.

2. The sports injury sensing system of claim 1, further comprising:
a plurality of inertial sensors, being connected to the sports injury sensing apparatus through a network, wherein each inertial sensor is installed on one of the body parts of the user to generate the inertial sensing data of each body part; and
an infrared thermal imager sensor, being connected to the sports injury sensing apparatus through the network, and being configured to generate the thermal image sensing datum.

3. The sports injury sensing system of claim 1, wherein the sports injury sensing apparatus further performs following operations:
receiving an input datum, wherein the input datum comprises an exercise type and exercise intensity; and
determining the exercising body part based on the input datum and the inertial sensing data.

4. The sports injury sensing system of claim 3, wherein the sports injury sensing apparatus further performs following operations:
generating a joint angle range and a movement trajectory range based on the exercise type and the exercise intensity;
calculating a joint angle and a movement trajectory based on the inertial sensing data; and
comparing whether the joint angle and the movement trajectory fall within the joint angle range and the movement trajectory range to determine whether to generate a danger warning message.

5. The sports injury sensing system of claim 3, wherein the sports injury sensing apparatus further performs following operations:
generating a muscle temperature range based on the exercise type and the exercise intensity; and
comparing whether the thermal image sensing datum falls within the muscle temperature range to determine whether to generate a danger warning message.

6. The sports injury sensing system of claim 1, further comprising:
a heart rate sensor, being connected to the sports injury sensing apparatus through a network, and being configured to sense a heart rate state of the user to generate a heart rate signal; and
wherein the sports injury sensing apparatus further determines whether the heart rate state of the user falls within a safe heart rate range according to the heart rate signal, and generates a danger warning message when the heart rate state is not within the safe heart rate range.

7. The sports injury sensing system of claim 6, wherein the sports injury sensing apparatus further performs following operations:
calculating the sports injury assessment based on the inertial sensing data, the temperature change of surrounding muscles and the heart rate signal.

8. A sports injury sensing method, being adapted for use in a sports injury sensing system, the sports injury sensing system comprising a sports injury sensing apparatus, the sports injury sensing method being performed by the sports injury sensing apparatus and comprising following steps:
receiving a plurality of inertial sensing data of a user, wherein each of the inertial sensing data corresponds to a body part of the user;
determining an exercising body part of the user based on the inertial sensing data;
receiving a thermal image sensing datum corresponding to the exercising body part, wherein the thermal image sensing datum indicates a body temperature state of the user;
analyzing a temperature change of surrounding muscles of the exercising body part based on the thermal image sensing datum; and
calculating a sports injury assessment based on the inertial sensing data and the temperature change of surrounding muscles.

9. The sports injury sensing method of claim 8, wherein the sports injury sensing system further comprises:
a plurality of inertial sensors, being connected to the sports injury sensing apparatus through a network, wherein each inertial sensor is installed on one of the body parts of the user to generate the inertial sensing data of each body part; and
an infrared thermal imager sensor, being connected to the sports injury sensing apparatus through the network, and being configured to generate the thermal image sensing datum.

10. The sports injury sensing method of claim 8, wherein the sports injury sensing method further comprises following steps:
receiving an input datum, wherein the input datum comprises an exercise type and exercise intensity; and
determining the exercising body part based on the input datum and the inertial sensing data.

11. The sports injury sensing method of claim 10, wherein the sports injury sensing method further comprises following steps:
generating a joint angle range and a movement trajectory range based on the exercise type and the exercise intensity;
calculating a joint angle and a movement trajectory based on the inertial sensing data; and
comparing whether the joint angle and the movement trajectory fall within the joint angle range and the movement trajectory range to determine whether to generate a danger warning message.

12. The sports injury sensing method of claim 10, wherein the sports injury sensing method further comprises following steps:
generating a muscle temperature range based on the exercise type and the exercise intensity; and
comparing whether the thermal image sensing datum falls within the muscle temperature range to determine whether to generate a danger warning message.

13. The sports injury sensing method of claim 8, wherein the sports injury sensing system further comprises:
a heart rate sensor, being connected to the sports injury sensing apparatus through a network, and being configured to sense a heart rate state of the user to generate a heart rate signal; and
wherein the sports injury sensing apparatus further determines whether the heart rate state of the user falls within a safe heart rate range according to the heart rate signal, and generates a danger warning message when the heart rate state is not within the safe heart rate range.

14. The sports injury sensing method of claim 13, wherein the sports injury sensing method further comprises following steps:
calculating the sports injury assessment based on the inertial sensing data, the temperature change of surrounding muscles and the heart rate signal.
